(19) United States
(12) Patent Application Publication
(10) Pub. No.: US 2019/0008764 A1
(43) Pub. Date: Jan. 10, 2019

(54) SERUM EYE DROP FORMULATIONS

(71) Applicant: HV Pharmacy, Inc., Cockeysville, MD (US)

(72) Inventor: Timothy E. ASKEW, Baltimore, MD (US)

(73) Assignee: HV Pharmacy, Inc.

(21) Appl. No.: 16/027,984

(22) Filed: Jul. 5, 2018

Related U.S. Application Data
(60) Provisional application No. 62/528,875, filed on Jul. 5, 2017.

(51) Int. Cl. A61K 9/00

(52) U.S. Cl. CPC A61K 9/0048

(57) ABSTRACT

Enhanced serum eye drops and methods for making the enhanced serum eye drops obtaining a volume of serum suitable for a given patient and incorporate an exogenous active ingredient into the volume of serum. The active ingredient is intended to yield a positive clinical impact on an ocular ailment associated with the given patient.

### CROSS-REFERENCE TO RELATED APPLICATIONS

The present application claims priority to U.S. Provisional Patent Application No. 62/528,875, the entire contents of which is incorporated herein by reference.

### TECHNICAL FIELD

Embodiments of the subject matter disclosed herein relate to serum eye drops.

### BACKGROUND

Human tears have antimicrobial, nourishing, mechanical, and optical properties. In addition, human tears contain components such as nerve and epidermal growth factors, fibronectin, and vitamins to support proliferation, migration, and differentiation of the corneal and conjunctival epithelium. A lack of these epitheliotropic factors, for example, in dry eye, can result in severe ocular surface disorders such as persistent epithelial defects that may lead to a loss of sight. The use of autologous serum eye drops or allogeneic serum eye drops, collectively serum eye drops (SED), has been reported in peer-reviewed clinical literature and is known as an effective treatment for severe ocular surface disorders.

Typically, SEDs are produced from donated whole blood and are non-allergenic. The biomechanical and biochemical properties of SEDs are similar to normal tears. Cell culture experiments performed in vitro showed that corneal epithelial cell morphology and function are better maintained by SEDs than by pharmaceutical tear substitutes. Clinical cohort studies have reported SEDs successfully used for severe dry eyes, severe ocular surface disorders, and persistent epithelial defects.

While broad consensus exists on the use of SEDs for chronic ocular surface disorders, there is little consensus on formulations including serum concentrations and diluents, preparation methodologies, storage procedures, and treatment protocols including patient dosing protocols.

### SUMMARY

Exemplary embodiments are directed to methods for making enhanced serum eye drop formulations. A patient to receive an enhanced serum eye drop formulation is identified. Identifying the patient to receive the enhanced serum eye droop formulation includes identifying diseases of the eye associated with the patient and states of the identified diseases of the eye. At least one active ingredient suitable for treating at least one of the identified diseases of the eye is identified. Identifying the active ingredient includes identifying an active ingredient suitable for treating a given eye disease at the identified current state of that eye disease. Identifying the active ingredient also includes determining an appropriate therapeutic level of the active ingredient in the enhanced serum eye drop formulation. At least one suitable diluent to be used for the enhanced serum is identified. The identified diluent is compatible with the identified active ingredient. A determination is made regarding the desired percentage of volume of diluent to volume of serum in the enhanced serum eye drop formulation.

Having identified the constituents of the enhanced SED formulation, whole blood suitable for the patient is obtained, either from the patient or a compatible donor, and serum is recovered or separated from the whole blood. The serum and diluent are combined according to the determined volume of diluent to volume of serum. The active ingredient, at the identified therapeutic level, is added to at least one of the whole blood, the serum and the combined serum and diluent.

A determination is made regarding whether the whole blood, the whole blood combined with the active ingredient, the serum, the serum combined with the active ingredient, the combined serum and diluent or the combined serum and diluent combined with the active ingredient is to be filtered.

This includes, for example, filtering at less than or equal to 22 microns. As the enhanced SED utilizes sterile constituents, filtering at less than or equal to 22 microns is not required. In one embodiment, filtering is conducted at greater than or equal to 45 microns to remove any debris. Filtering, however, is optional, and an enhanced SED can be created without any filtering. The enhanced serum eye drop formulation is packaged and provided to the patient. In one embodiment, the active ingredient is added to the combined serum and diluent in the packaging, e.g., for active ingredients that exist as mixtures or do not otherwise go into solution. In one embodiment, the enhanced SED formulations are not filtered following the addition of active ingredients existing as mixtures.

Exemplary embodiments are directed to a method for making enhanced serum eye drops. A given patient is identified, and at least one ocular ailment associated with the given patient is identified. In one embodiment, at least one of an ocular disease, an ocular disease state and an ocular condition is identified. A volume of serum suitable for a given patient is obtained. In one embodiment, a volume of autologous serum or obtaining a volume of allogeneic serum is obtained.

An exogenous active ingredient suitable to treat the identified ocular ailment is identified. In addition, a therapeutic concentration of the active ingredient in the serum eye drops sufficient to deliver the positive clinical impact is determined. In one embodiment, the therapeutic concentration is from about 0.001 mg/ml to about 5 mg/ml. The exogenous active ingredient is incorporated into the volume of serum. The exogenous active ingredient is intended to yield a positive clinical impact on an ocular ailment associated with the given patient.

In one embodiment, the active ingredient is a chemically active ingredient or a biologically active ingredient. Suitable active ingredients include an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent and combinations thereof.

In one embodiment, the serum is mixed with a diluent. The diluent is compatible with the active ingredient. Suitable diluents include a balanced salt solution, artificial tears and a combination thereof. In one embodiment, the serum is mixed with the diluent at a volume ratio of 20% to 50% serum to diluent. In one embodiment, the active ingredient is added to the diluent before mixing the serum with the diluent. In one embodiment, the volume of serum is obtained from whole blood, and at least one of the whole blood, the serum, the diluent, the serum mixed with the diluent, the serum containing the active ingredient, the serum mixed with the diluent and containing the active ingredient and the serum eye drops is filtered. In one embodiment, a filter having a pore size sufficient to generate sterilized filtrate is used. In one embodiment, a filter having a pore size greater than or equal to about 0.45 microns is used.

Exemplary embodiments are also directed to method for making enhanced serum eye drops where a plurality of ocular ailments associated a given patient is identified, and a plurality of exogenous active ingredients intended to yield a positive clinical impact on each identified ocular ailment is identified. A volume of serum suitable for the given patient is obtained, and the serum is mixed with a diluent compatible with each active ingredient. Each active ingredient is incorporated into the volume of serum and diluent.

Identifying the plurality of active ingredients includes at least one of identifying desired beneficial and symbiotic interactions among the active ingredients, identifying optimum concentrations of the active ingredients and identifying if each active ingredient is to be included in the enhanced serum eye drops as a mixture, in suspension, in solution or as an emulsion. In one embodiment, a plurality of exogenous active ingredients is identified from a group containing an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent and combinations thereof. In addition, a volume of autologous serum or a volume of allogeneic serum is obtained. The serum is mixed with a diluent that is a balanced salt solution, artificial tears or a combination thereof.

Exemplary embodiments are also directed to enhanced serum eye drops containing a volume of serum suitable for a given patient and an exogenous active ingredient. The active ingredient is intended to yield a positive clinical impact on an ocular ailment associated with the given patient. In one embodiment, the serum eye drops include a diluent. The diluent is compatible with the active ingredient and is present in the serum eye drops at a volume ratio of 20% to 50% serum to diluent.

### BRIEF DESCRIPTION OF THE DRAWINGS

The accompanying drawings, which are incorporated in and constitute a part of the specification, illustrate a plurality of embodiments and, together with the following descriptions, explain these embodiments.

FIG. 1 is a schematic representation of an embodiment of a system for use in creating enhanced SED formulations; and

FIG. 2 is a flow chart illustrating an embodiment of a method for creating enhanced SED formulations.

### DETAILED DESCRIPTION

The following description of the embodiments refers to the accompanying figures. The same reference numbers in different figures identify the same or similar elements. Reference throughout the whole specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, particular features, structures or characteristics may be combined in any suitable manner in one or more embodiments.

Exemplary embodiments are directed to enhanced serum eye drops (SED) or enhanced SED formulations that include one or more active ingredients. An active ingredient is any ingredient intended to deliver a positive clinical impact on an ocular ailment associated with a patient. As used herein, an ocular ailment refers to any ocular disease, any ocular disease state, any ocular syndrome, any ocular disorder and any ocular condition. Exemplary embodiments are also directed to methods for making these enhanced SED formulations and therapeutic uses for these enhanced SED formulations. The enhanced SED formulations include patient specific enhanced formulations, ocular disease specific enhanced formulations, ocular disease state specific enhanced formulations and ocular condition specific enhanced formulations.

Patients suffering from ocular ailments such as dry eye syndrome are prescribed multiple medications to manage the underlying disease state and the symptoms. For example, a given patient is provided with separate prescriptions for SEDs and cyclosporine. Exemplary embodiments provide patients with a therapeutic eye drop, i.e., the enhanced SED formulation, containing both SED and cyclosporine. The enhanced SED formulation makes it easier for patients to comply with the physician's prescribed eye drop regimen. In addition, waste during the application, storage and expiration process is reduced, and the combined eye drop, i.e., the enhanced SED formulation, reduces costs to the patient by eliminating the need for multiple separate custom prescriptions.

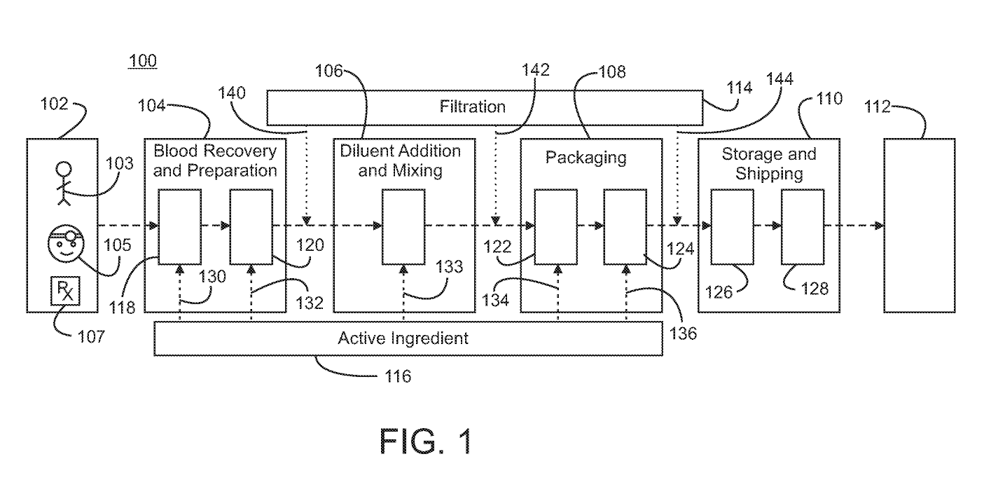

Referring to FIG. 1, an exemplary embodiment of a system 100 for producing enhanced SED formulations is illustrated. The various parts illustrated can be viewed as parts or modules of the system for making enhanced SED formulations. The system includes a plurality of modules that perform different aspects of the formulation, creation and delivery of the enhanced SEDs. The system includes a formulation input and creation module 102. The patient input and formulation creation module obtains the data and information necessary to create a particular or a given enhanced SED. This information includes an identification of the patient 103 or patients for whom the enhanced SED is to be created. Identification of the given patent includes ocular ailments that are to be treated or targeted by the enhanced SED, general health and medical history of the given patient, current medications taken or used by the given patient, known patient allergies and any know clinical history of SED use by the given patient. The patient input and formulation creation module also receives input from third party sources including, for example, physicians 105, pharmacies 107, pharmacologists and pharmacists.

The patient information and other data are used by the patient input and formulation creation module to formulated and appropriate enhanced SED. The enhanced SED formulation includes the type of serum to be obtained, for example, autologous serum or allogeneic serum. In one embodiment, the enhanced SED is a generic formulation that is suitable for use with multiple patients. The enhanced SED formulation also includes and identification of the ocular ailments to me treated, the active ingredients to be used to target those ocular ailments and the diluents to be included in the enhanced SED. The enhanced SED formulation also identifies any additional components including, for example, additional inactive ingredients.

In one embodiment, the identified active ingredients target or address patient specific ocular ailments including the current ocular disease state and ocular conditions. These active ingredients include chemically active ingredients and biologically active ingredients that either individually or in combination have a positive clinical impact on the identified ocular ailments. Suitable active ingredients include, but are not limited to, immunosuppressants, non-steroidal anti-inflammatory agents (NSAID), hormones, biologically derived active ingredients, steroids, antibiotics, anti-fungals (i.e., antimycotics, fungicides and fungistatics), anticholinergics, mucolytics, chemotherapeutic agents and combinations thereof.

In one embodiment, a single active ingredient is identified, for example, from a single type of active ingredient, e.g., immunosuppressants. In one embodiment, active ingredients are identified from each of two or more types of active ingredients. For example, a single active ingredient is identified from each of two of more types of active ingredients. In one embodiment, a plurality of active ingredients from a single type of active ingredient are identified. In one embodiment, a plurality of active ingredients from each of two or more types or categories of active ingredients, e.g., multiple hormones or multiple immunosuppressants, are identified.

In one embodiment, the patient input and formulation creation module identifies desired beneficial or symbiotic interactions among active ingredients, optimum concentrations of the active ingredients, a state in which each active ingredient exists within the enhanced SED formulation, e.g., mixture, suspension, solution or emulsion. In one embodiment, the patient input and formulation creation module identifies the appropriate diluent or diluents to be included in the enhanced SED formulation. Suitable diluents include, but are not limited to, balanced salt solutions, artificial tears and combinations thereof.

Having formulated the desired enhanced SED, the patient input and formulation module identified methodologies and steps for utilizing the additional modules of the system 100 for producing enhanced SED formulations in order to create the formulated enhanced SED.

The system includes a blood recovery and preparation module 104. At this module serum that is suitable for use by the identified given patient or patients is obtained. In general, the blood recovery and preparation module is where whole blood is obtained and serum is separated from the whole blood in accordance with the enhanced SED formulated by the patient input and formulation creation module. The blood recovery and preparation module includes a whole blood acquisition module 118. At the whole blood acquisition module, whole blood is obtained or drawn from an appropriate donor, e.g., the given patient or a compatible third-party donor.

The blood recovery and preparation module also includes a serum separation module 120 where serum is recovered or separated from the whole blood. Suitable methods for separating serum from whole blood are known and available in the art. In one embodiment, separation of serum from whole blood includes coagulating the whole blood at a prescribed temperature for a prescribed period of coagulation time and centrifugation of the coagulated whole blood at a prescribed revolutions per minute (RPM) for a prescribed period of centrifugation time. In one embodiment, the prescribed temperature, the prescribed period of coagulation time, the prescribed RPM and the prescribed period of centrifugation time are consistent, i.e., the same, from batch to batch of a given enhanced SED formulation and from enhanced SED formulation to enhanced SED formulation. Standardization of serum separation techniques and conditions yields a consistent quality in the enhanced SED, regardless of when a given particular enhanced SED formulation is made or of changes in the formulation of the enhanced SED.

The system includes a diluent addition and mixing module 106 where the identified diluent or diluents are added to the serum to achieve the desired volume percentages of serum to diluent. In one embodiment, the desired volume percentages ranged from about 20% volume of serum/volume of diluent to about 50% volume of serum/volume of diluent. The blood recovery and preparation module is also where the combined diluent and serum are mixed. Suitable mixing methods include, but are not limited to, using a hand vortex mixer.

The system includes a packaging module 108. The packaging module includes a package filling module 122 where the desired type of packaging is filled with the combined serum and diluent, and any other ingredients. In addition, the packaging module includes a package sealing module 124 where the filled package is sealed. Suitable types of packaging include, but are not limited to, dropper bottles.

The system includes an active ingredient addition module 116 where the identified active ingredients are added. The active ingredient addition module is in communication the blood recovery and preparation module, the diluent addition and mixing module and the packaging module, including the sub-modules contained within these modules. Therefore, the active ingredient addition module facilitates adding the desired and identified active ingredients at one or more of a plurality of points during creation of the enhanced SED formulation. The plurality of points includes a whole blood addition point 130, a serum addition point 132, a combined diluent and serum addition point 133, which includes adding active ingredients concurrent with adding serum to the diluent, a packing filling addition point 134 and a package sealing addition point 136.

In one embodiment, the formulation input and creation module determines the addition point or addition points for adding the active ingredients. This determination is based on at least one of the form of the active ingredient, e.g., powder or liquid, the solubility of the active ingredient in whole blood, serum or diluent and effects of steps performed in subsequent modules on the efficacy of the active ingredient, e.g., serum separation or diluent mixing.

The system includes a filtration module 114 where the SED or various ingredients of the SED are filtered. The filtration module is in communication the blood recovery and preparation module, the diluent addition and mixing module. Therefore, the filtration module facilitates filtering the enhanced SED formulation or components of the enhanced SED formulation at one or more of a plurality of points during creation of the enhanced SED formulation. The plurality of points includes a point for filtering the whole blood 140, a point for filtering the serum 142, and a point for filtering the combined serum and diluent 144. Filtering can be used at single point or multiple points. In addition, at any point at which filtering is applied, the liquid filtered can include one or more of the active ingredients or inactive ingredients. In one embodiment, filtering is not used.

Suitable types of filters include, but are not limited to, tortuous path filters and filters with pores of the desired size. Suitable materials for the filters include, but are not limited to, hydrophilic, hydrophobic, oleophilic and oleophobic materials. The filter materials are selected based on the given step for filtering and the current state of the enhanced SED formulation, e.g., whole blood, serum or serum and diluent. In one embodiment, filtering is used to sterilize the liquid being filtered. Therefore, filtering is conducted with a filter having a pore size sufficient to create a sterilized filtrate, e.g., a pore size less than or equal to 22 microns. Filtering with a pore size less than or equal to 22 microns achieves two benefits, removal of debris that could cause inflammation in the eye and sterilization of the preparation according to United States Pharmacopeial Convention (USP) 797. In another embodiment, filtering is conducted at a pore size equal to or greater than about 0.45 microns. This filter allows for easier filtering, removes the debris, but does not sterilize the preparation. Filtering the combined serum and diluent, i.e., the SED, either with or without active ingredients, e.g., 10.0 mg/ml cyclosporine, using a filter having a maximum pore size rating of 22 micron, enhances the quality of the final enhanced SED formulation. In one embodiment, filtering is not used.

The system also includes a delivery and administering module 112 where the enhanced SED formulation is delivered to the patient for use or administration.

Exemplary embodiments are also directed to the enhanced SED formulations. In one embodiment, the enhanced serum eye drops include a volume of serum suitable for a given patient. Serum that is suitable for the patient is compatible with the patient and includes a volume of autologous serum or a volume of allogeneic serum. In one embodiment, the serum is a generic volume that is compatible with two or more patients.

The enhanced SED also includes at least one exogenous active ingredient. The active ingredient is exogenous as it is not native to the serum and is added to the serum. Each active ingredient yields a positive clinical impact on an ocular ailment associated with the given patient. The active ingredient includes chemically active ingredients and biologically active ingredients. Suitable active ingredients include, but at not limited to, an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent and combinations thereof.

Suitable immunosuppressants include, but are not limited to, cyclosporine and tacrolimus. In one embodiment, the immunosuppressants are present in the enhanced SED in concentrations ranging from 0.001 mg/ml to 100.00 mg/ml. In another embodiment, the immunosuppressants are present in concentrations ranging from 0.01 mg/ml to 3.00 mg/ml. Suitable NSAIDs include, but are not limited to, ketorolac and bromfenac. In one embodiment, the NSAIDs are present in the enhanced SED in concentrations ranging from 0.01 mg/ml to 25.00 mg/ml. In one embodiment, the NSAIDs are present in concentrations ranging from 0.001 mg/ml to 10.00 mg/ml. Suitable hormones include, but are not limited to, testosterone, progesterone, estradiol, and estriol. In one embodiment, hormones are present in the enhanced SED in concentrations ranging from 0.001 mg/ml to 100.00 mg/ml. In one embodiment, hormones are present in concentrations ranging from 0.01 mg/ml to 2.00 mg/ml.

Suitable biologically derived active ingredients include, but not limited to, bevacizumab, ranibizumab, and aflibercept. In one embodiment, biologically derived active ingredients are present in the enhanced SED in concentrations ranging from 0.001 mg/ml to 100.00 mg/ml. In one embodiment, biologically derived active ingredients are present in the enhanced SED in concentrations ranging from 0.01 mg/ml to 20.00 mg/ml. Suitable steroids include, but are not limited to, prednisolone and dexamethasone. In one embodiment, steroids are present in the enhanced SED in concentrations ranging from 0.01 mg/ml to 50.00 mg/ml. In one embodiment, steroids are present in the enhanced SED in concentrations ranging from 0.01 mg/ml to 10.00 mg/ml. Suitable antibiotics include, but are not limited to, ofloxacin, ciprofloxacin, and moxifloxacin. In one embodiment, antibiotics are present in the enhanced SED in concentrations ranging from 0.001 mg/ml to 20.00 mg/ml. In one embodiment, antibiotics are present in the enhanced SED in concentrations ranging from 0.01 mg/ml to 1.00 mg/ml.

In one embodiment, vancomycin is present in the enhanced SED in an amount of from about 0.1 to about 100 mg/ml, preferably from about 12.5 mg/ml to about 50 mg/ml, of enhanced serum eye drops. In one embodiment, tobramycin is present in an amount of from about 0.1 to about 25 mg/ml, preferably from about 3 mg/ml to about 15 mg/ml, of enhanced serum eye drops. In one embodiment, cefazolin is present in an amount of from about 0.1 to about 100 mg/ml, preferably from about 12.5 mg/ml to about 50 mg/ml, of enhanced serum eye drops. In one embodiment, ceftazidime is present in an amount of from about 0.1 to about 100 mg/ml, preferably from about 12.5 mg/ml to about 50 mg/ml, of enhanced serum eye drops.

Suitable fungicides or fungistatics include, but are not limited to, voriconazole and amphotericin B, and are present in the enhanced SED in concentrations ranging from 0.001 mg/ml to 50.00 mg/ml. In one embodiment, the fungicides and fungistatics are present in the enhanced SED in concentrations ranging from 0.01 mg/ml to 2.00 mg/ml. Suitable anticholinergics include, but are not limited to, atropine and are present in the enhanced SED in concentrations ranging from 0.001 mg/ml to 2.00 mg/ml. In one embodiment, the anticholinergics are present in the enhanced SED in concentrations from 0.01 mg/ml to 2.00 mg/ml.

Suitable mucolytics include, but are not limited to, acetylcysteine and are present in the enhanced SED in concentrations ranging from 1.00 mg/ml to 200.00 mg/ml. In one embodiment, the mucolytics are present in the enhanced SED in concentrations ranging from 5.00 mg/ml to 100.00 mg/ml. Suitable chemotherapeutic agents, i.e., one or more anti-cancer drugs, otherwise known as non-specific intracellular poisons, include, but are not limited to, fluorouracil, mitomycin, and interferon. In one embodiment, the chemotherapeutic agents are present in the enhanced SED in concentrations ranging from 0.001 mg/ml to 5.00 mg/ml. In one embodiment, the chemotherapeutic agents are present in the enhanced SED in concentrations ranging from 0.01 mg/ml to 2.00 mg/ml

In one embodiment, the enhanced serum eye drops also include one or more diluents. Each diluent is compatible with the active ingredient and present in the serum eye drops to achieve a desired volume ratio with the serum. Suitable volume ratios include a volume ratio of about 0.10% to about 99.9% of serum to diluent. In one embodiment, the volume ratio is from about 15% to about 55% of serum to diluent. Suitable diluents include, but are not limited to, balanced salt solutions and artificial tears.

Suitable balanced salt solutions include, but are not limited to, 0.9% sodium chloride solutions. In one embodiment, the balanced salt solution is BSS® Sterile Irrigating Solution, commercially available from Alcon Laboratories, Inc. of Fort Worth, Tex. BSS® Sterile Irrigating Solution is a sterile balanced salt solution, each ml containing sodium chloride (NaCl) 0.64%, potassium chloride (KCl) 0.075%, calcium chloride dihydrate (CaCl₂.2H₂O) 0.048%, magnesium chloride hexahydrate (MgC1₂.6H₂O) 0.03%, sodium acetate trihydrate (C₂H₃NaO₂.3H₂O) 0.39%, sodium citrate dihydrate (C₆H₅Na₃O₇.2H₂O) 0.17%, sodium hydroxide and/or hydrochloric acid (to adjust pH), and water for injection. In one embodiment, the pH is approximately 7.5. In one embodiment, the osmolality is approximately 300 mOsm/Kg.

Suitable artificial tears contain polyvinyl alcohol, glycerin, carboxymethylcellulose sodium, povidone, polyethylene glycol, and hypromellose, among other ingredients. In one embodiment, artificial tears are present in the enhanced SED formulation in concentrations ranging from about 0.001% to about 50.00%. In one embodiment, artificial tears are present in the enhanced SED in concentrations ranging from about 0.01% to about 5.00%.

In one embodiment, the enhanced SED formulation includes a 20% V/V to 50% V/V SED, i.e., serum to appropriate diluent or diluents, and 0.10 mg/ml to 10.0 mg/ml cyclosporine. In another embodiment, the enhanced SED formulation includes a 50% V/V SED, serum and diluent, and 10.0 mg/ml cyclosporine.

Exemplary embodiments of enhanced SED formulations have been independently tested by an independent FDA-registered testing laboratory for stability. In general, the enhanced SED formulations exhibit improved stability. For example, enhanced SED formulations containing cyclosporine mixed with a 20% V/V and 50% V/V SED solution were stable for 38 days. Stability is defined as the active ingredient maintaining 90% to 110% of its mg/ml initial potency over a given period, i.e., 38 days.

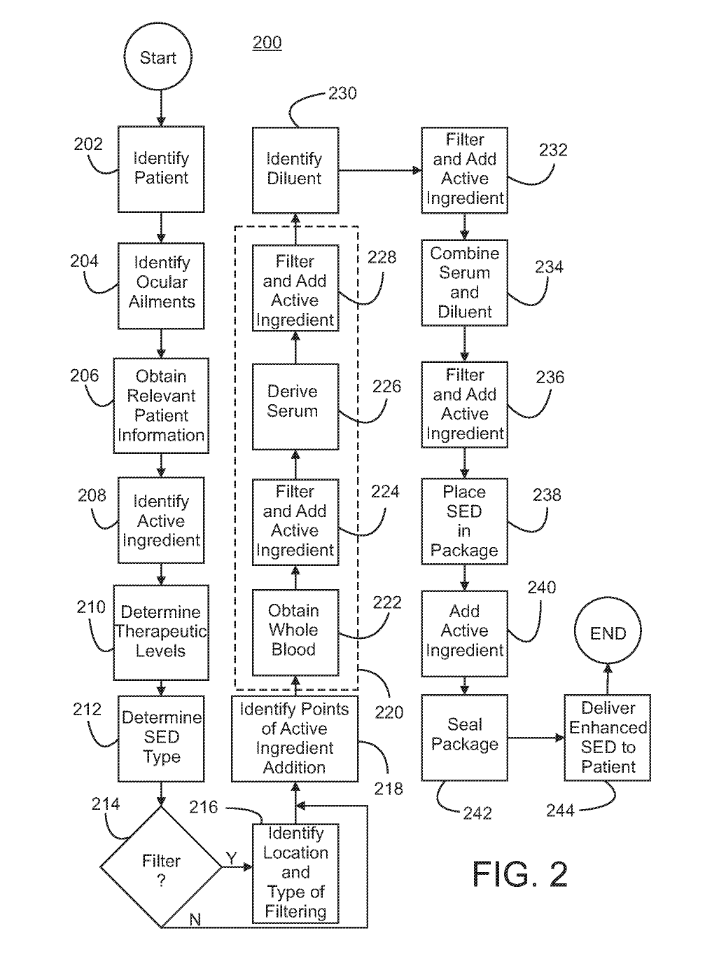

Referring now to FIG. 2, exemplary embodiments are also directed to a method for making enhanced serum eye drops 200. The given patient for whom the enhanced SED is being created is identified 202. In one embodiment a single patient is identified. Alternatively, multiple patients are identified. At least one ocular ailment associated with the patient is identified 204. Identification of an ocular ailment includes identifying at least one of an ocular disease, an ocular disease state and an ocular condition associated with the patient. In one embodiment, a plurality of ocular ailments associated a given patient are identified. In one embodiment, additional relevant patient information is obtained 206. This additional information includes, for example, medical history, allergies and current medications.

At least one exogenous active ingredient suitable to treat the identified ocular ailment is identified 208. An active ingredient is intended to treat an ocular ailment if that active ingredient yields a positive clinical impact on the ocular ailment. In one embodiment, a plurality of active ingredients suitable to treat the identified ocular ailment is identified. When a plurality of ocular ailments has been identified for the given patient, a plurality of exogenous active ingredients suitable to yield a positive clinical impact on each identified ocular ailment is identified. When multiple active ingredients are identified, identification of the active ingredients includes considering interactions among the multiple active ingredients. The active ingredients include chemically active ingredients and biologically active ingredients. Suitable active ingredients are discussed herein and include, but are not limited to, an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent or combinations thereof.

In one embodiment, a therapeutic concentration of each identified active ingredient in the enhanced serum eye drops is identified 210. The therapeutic concentrated is sufficient to deliver the positive clinical impact. Suitable therapeutic concentrations are discussed herein and include about 0.001 mg/ml to about 5 mg/ml.

The type of enhanced SED is then determined 212. Suitable types of enhanced SED formulations include autologous and allogeneic. In one embodiment, the type of SED formulation is generic, i.e., suitable for two or more patients. A suitable SED is an SED that is compatible for use on a given patient and includes SED formulations that are derived from genetically identical, genetically dissimilar, immunologically compatible and immunologically incompatible whole blood.

A determination is then made regarding whether the enhanced SED or any of the components used to create the SED are to be filtered 214. If filtering is to be used, then the location and type of filtering are identified 216. Identifying the type of filtering includes identifying the type of filter, e.g., tortuous path, absolute, etc., and the pore size of that filter. In one embodiment, filtering is identified that is suitable to produce sterile filtrate, e.g., less than about 0.22 microns. In another embodiment, filtering is identified having a pore size equal to or greater than about 0.45 microns. The type of filtration is identified for each location or constituent to be filtered. These locations and constituents include at least one of the whole blood, the serum, the diluent, the serum mixed with the diluent, the serum containing the active ingredient, the serum mixed with the diluent and containing the active ingredient and the serum eye drops.

Having determined the desired filtration regiment or if no filtering is to be used, the points of addition of each active ingredient are identified 218. The active ingredient can be added to the whole blood, the serum, the diluent, a mixture of the diluent and serum, and the SED during packaging. Identification of points of addition of the active ingredient includes identifying the state in which the active ingredient will be added, e.g., solution, mixture, emulsion, suspension, determining if the active ingredient is added before or after any filtering and identifying any specific mixing requirements, e.g., shaking or vortex mixers.

A volume of serum suitable for the identified patient and in accordance with the type of enhanced SED is obtained 220. In one embodiment, the serum is obtained from whole blood. In one embodiment, a volume of whole blood is obtained 216. This volume of whole blood can be obtained from the patient, for a volume of autologous serum, or from a suitable third-party donor, for a volume of allogeneic serum. Filtering and active ingredient addition to the whole blood is performed in accordance with any identified filtration and active ingredient addition requirements 224. The volume of serum is then derived from the whole blood 226. Suitable methods for deriving serum from whole blood are discussed herein and include any suitable method known and available in the art. Filtering and active ingredient addition to the serum is performed in accordance with any identified filtration and active ingredient addition requirements 228.

Any desired diluent to be mixed with the serum is identified 230. Suitable diluents are identified herein and include, but are not limited to, a balanced salt solution, artificial tears and combinations thereof. In general, the diluent is compatible with the active ingredients. Filtering and active ingredient addition to the diluent is performed in accordance with any identified filtration and active ingredient addition requirements 232. Therefore, the active ingredient is added to the diluent before mixing the serum with the diluent. The diluent is combined or mixed with the serum 234. In one embodiment, the serum is mixed with the diluent at a volume ratio of about 20% to about 50% serum to diluent. Filtering and active ingredient addition to the diluent and serum mixture is performed in accordance with any identified filtration and active ingredient addition requirements 236.

The resulting SED or mixture of serum and diluent is added to the packaging 238. If any active ingredients are to be added at this point, those active ingredients are added with the SED to the packaging 240. The package containing the enhanced SED is sealed 242 and delivered to the given patient 244.

Exemplary embodiments standardized the preparation of enhanced SED formulations and improve the finished quality of the enhanced SED formulations, including the preparation of the enhanced SED formulation by the system illustrated FIG. 1 and the method illustrated in FIG. 2, by ensuring chemical compatibility of diluents and active ingredients. Factors and method steps used to ensure this compatibility and to provide the desired enhancements include, blood draw methodology, blood coagulation time, blood coagulation temperature, serum separation via centrifuge, serum separation at a specific centrifugal RPM, serum separation at a specific centrifugal time, diluent or diluents selection, filter type and pore size selection, filter step selection or filtration timing, stock solution creation, stock solution filtration, storage container or packing selection, and storage temperature selection. As used herein, stock solution refers to any solution of any component or constituent that is made up at any time and used in multiple preparations of SED formulations.

In one embodiment, enhanced SED formulations are prepared in an ISO 5 clean room. Other variables include, but are not limited to, an amount of blood collected and an amount of serum yielded from the collected whole blood. In one embodiment, patients or blood donors are provided with instructions prior to obtaining the whole blood in order to affect the amount or quality of the resulting serum, e.g., instructing a patient or blood donor to drink or increase water intake to increase the yield of serum. In one embodiment, the collected blood or obtained serum or SED formulations are shipped using temperature validated containers to ensure 2°-8° C. storage conditions.

The foregoing written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

What is claimed is:

1. A method for making enhanced serum eye drops, the method comprising:
obtaining a volume of serum suitable for a given patient; and
incorporating an exogenous active ingredient into the volume of serum, the active ingredient intended to yield a positive clinical impact on an ocular ailment associated with the given patient.

2. The method of claim 1, wherein obtaining a volume of serum comprises obtaining a volume of autologous serum or obtaining a volume of allogeneic serum.

3. The method of claim 1, wherein the active ingredient comprises a chemically active ingredient or a biologically active ingredient.

4. The method of claim 1, wherein the active ingredient comprises an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent or combinations thereof.

5. The method of claim 1, wherein the method further comprises:
identifying the given patient;
identifying the ocular ailment associated with the given patient; and
identifying the active ingredient suitable to treat the identified ocular ailment.

6. The method of claim 5, wherein identifying the ocular ailment comprises identifying at least one of an ocular disease, an ocular disease state and an ocular condition.

7. The method of claim 5, wherein identifying the active ingredient comprises determining a therapeutic concentration of the active ingredient in the serum eye drops sufficient to deliver the positive clinical impact.

8. The method of claim 7, wherein the therapeutic concentration comprises about 0.001 mg/ml to about 5 mg/ml.

9. The method of claim 1, wherein the method further comprises mixing the serum with a diluent, the diluent compatible with the active ingredient.

10. The method of claim 9, wherein the diluent comprises a balanced salt solution, artificial tears or a combination thereof.

11. The method of claim 9, wherein mixing the serum with the diluent comprises mixing the serum with the diluent at a volume ratio of 20% to 50% serum to diluent.

12. The method of claim 9, wherein incorporating the active ingredient in the serum further comprises adding the active ingredient to the diluent before mixing the serum with the diluent.

13. The method of claim 9, wherein:
obtaining the volume of serum comprises obtaining the volume of serum from whole blood; and
the method further comprises filtering at least one of the whole blood, the serum, the diluent, the serum mixed with the diluent, the serum containing the active ingredient, the serum mixed with the diluent and containing the active ingredient and the serum eye drops.

14. The method of claim 13, wherein filtering further comprises using a filter having a pore size sufficient to generate sterilized filtrate.

15. The method of claim 13, wherein filtering further comprises using a filter having a pore size greater than or equal to about 0.45 microns.

16. A method for making enhanced serum eye drops, the method comprising:
identifying a plurality of ocular ailments associated a given patient;
identifying a plurality of exogenous active ingredients intended to yield a positive clinical impact on each identified ocular ailment;
obtaining a volume of serum suitable for the given patient;
mixing the serum with a diluent, the diluent compatible with each active ingredient; and
incorporating each active ingredient into the volume of serum and diluent.

17. The method of claim 16, wherein identifying the plurality of active ingredients comprises at least one of:
identifying desired beneficial and symbiotic interactions among the active ingredients;
identifying optimum concentrations of the active ingredients; and
identifying if each active ingredient is to be included in the enhanced serum eye drops as a mixture, in suspension, in solution or as an emulsion.

18. The method of claim 16, wherein:
identifying a plurality of exogenous active ingredients further comprises identifying a plurality of active ingredients from a group comprising an immunosuppressant, a non-steroidal anti-inflammatory agent, a hormone, a biologically derived active ingredient, a steroid, an antibiotic, a fungicide, a fungistatic, an anticholinergic, a mucolytic, a chemotherapeutic agent or combinations thereof;
obtaining a volume of serum further comprises obtaining a volume of autologous serum or obtaining a volume of allogeneic serum; and
mixing the serum with a diluent further comprises mixing the serum with a balanced salt solution, artificial tears or a combination thereof.

19. Enhanced serum eye drops comprising:
a volume of serum suitable for a given patient; and
an exogenous active ingredient, the active ingredient intended to yield a positive clinical impact on an ocular ailment associated with the given patient.

20. The enhanced serum eye drops of claim 19, wherein the serum eye drops further comprise a diluent, the diluent compatible with the active ingredient and present in the serum eye drops at a volume ratio of 20% to 50% serum to diluent.
